(19) United States
(12) Patent Application Publication
(10) Pub. No.: US 2017/0042312 A1
(43) Pub. Date: Feb. 16, 2017

(54) COSMETIC PRODUCT

(71) Applicants: Chia-Hsin CHU, Taoyuan City (TW); Mei-Hsin CHU, Taoyuan City (TW); Asia-Team Biotechnology Co., Ltd., Taoyuan City (TW)

(72) Inventor: Cheng-Hung SHIH, Taoyuan City (TW)

(21) Appl. No.: 15/234,860

(22) Filed: Aug. 11, 2016

(30) Foreign Application Priority Data
Aug. 13, 2015 (TW) 104126356

(51) Int. Cl. A45D 44/00; A61N 5/06

(52) U.S. Cl. CPC A45D 44/002; A61N 5/0616; A61N 2005/0647; A61N 2005/0662; A61N 2005/0645

(57) ABSTRACT

A cosmetic product includes a main body and a barrier unit. The main body includes a base layer, a skincare preparation that is dispersed in or formed on the base layer, and a fluorescent layer that is disposed on the base layer and that includes a fluorescent material emitting light with a predetermined wavelength after being excited by a light source. The barrier unit is disposed on the fluorescent layer of the main body for blocking light emitted from the light source and having a wavelength less than 380 nm or between 780 nm and 1100 nm from entering into the fluorescent layer and the base layer.

### CROSS-REFERENCE TO RELATED APPLICATION

This application claims priority of Taiwanese Patent Application No. 104126356 filed on Aug. 13, 2015.

### FIELD

The disclosure relates to a cosmetic product, more particularly to a cosmetic product including a fluorescent layer that emits light with a predetermined wavelength after being excited by a light source.

### BACKGROUND

Light therapy has been widely used for cosmetic purposes, and involves exposure to specific wavelengths of light. It has been shown that exposure to light with wavelengths of 570 and 630 nanometers (nm) influence the fibroblasts within the dermis, which can further induce a restructuring of collagen fibers within the dermis so that skin rejuvenation can be achieved. Blue light acne treatment is a non-invasive procedure that uses light in the blue wavelength range of 400 nm to 420 nm to produce Coproporphyrin III and exhibit bactericidal effects on Propionibacterium. However, equipment used in light therapy is expensive, and medical procedures involving light therapy need to be conducted by trained professionals.

A cosmetic facial mask prepared by impregnating a nonwoven fabric with a cosmetic liquid such as a skin lotion, a serum and a milky lotion has been widely used so far. However, such types of facial masks often cannot achieve the same results as light therapy.

### SUMMARY

Therefore, an object of the disclosure is to provide a cosmetic product that can alleviate at least one of the drawbacks of the prior art.

According to the disclosure, the cosmetic product includes a main body and a barrier unit.

The main body includes a base layer, a skincare preparation that is dispersed in or formed on the base layer, and a fluorescent layer that is disposed on the base layer and that includes a fluorescent material emitting light with a predetermined wavelength after being excited by a light source.

The barrier unit is disposed on the fluorescent layer of the main body for blocking light emitted from the light source and having a wavelength less than 380 nm or between 780 nm and 1100 nm from entering into the fluorescent layer and the base layer.

### BRIEF DESCRIPTION OF THE DRAWINGS

Other features and advantages of the disclosure will become apparent in the following detailed description of the embodiment (s) with reference to the accompanying drawings, of which

FIG. 1 is a sectional view illustrating a first embodiment of a cosmetic product according to the disclosure;

FIG. 2 is a sectional view illustrating a second embodiment of the cosmetic product according to the disclosure; and

FIG. 3 is a sectional view illustrating a third embodiment of the cosmetic product according to the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

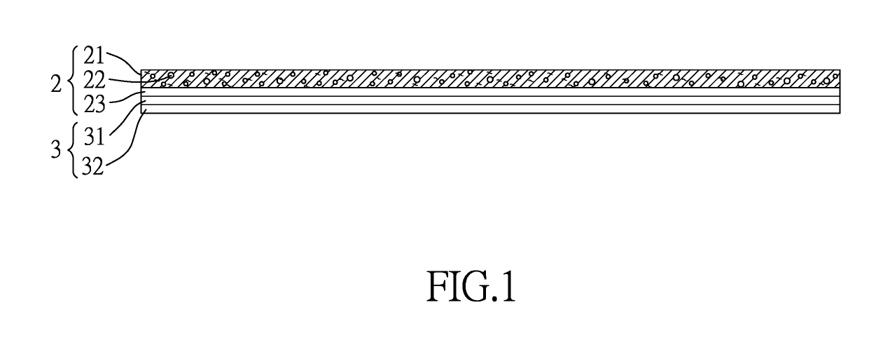

FIG. 1 illustrates a first embodiment of a cosmetic product according to this disclosure that includes a main body 2 and a barrier unit 3.

The main body 2 includes a base layer 21, a skincare preparation 22 that is dispersed in or formed on the base layer 21, and a fluorescent layer 23 that is disposed on and in contact with the base layer 21 and that includes a fluorescent material. The fluorescent material emits light with a predetermined wavelength after being excited by a light source (not shown).

To be specific, the base layer 21 may be made from a material selected from the group consisting of woven fabric and non-woven fabric. The form of the skincare preparation 22 has no specific limitations. For instance, if the skincare preparation 22 is in a liquid form, the skincare preparation 22 may be dispersed in and absorbed by the base layer 21. If the skincare preparation 22 is in a colloidal form, the skincare preparation 22 may be formed on one side of the base layer while the fluorescent layer 23 is disposed on and in contact with the other side of the base layer 21. In this embodiment, the skincare preparation 22 is in the liquid form and dispersed in the base layer 21. It should be noted that, based on the intended application and requirements of the cosmetic product, the skincare preparation 22 may include any appropriate ingredients.

Selection of the fluorescent material is also based on the intended application and requirements of the cosmetic product, e.g., specific skincare or cosmetic effect(s) to be induced by the predetermined wavelength of the light emitted from the fluorescent material. It should be noted that, the fluorescent material must be a non-toxic material, such as a non-toxic fluorescence dye, and the light emitted therefrom should be beneficial to human skin. In certain embodiments, the non-toxic fluorescence dye is a non-azo fluorescence dye. In certain embodiments, formation of the fluorescent layer 23 may be conducted by printing, dipping or other procedures.

When the light emitted from the fluorescent material has the predetermined wavelength ranging from 450 nm to 495 nm (blue light), the light of the fluorescent material may inhibit the inflammatory lesions and the secretions of the sebaceous gland, thereby reducing acne and the number of pimples, and promoting tissue repair. When the light emitted from the fluorescent material has the predetermined wavelength ranging from 495 nm to 560 nm (green light), the light of the fluorescent material may facilitate muscle growth and disinfection of the skin tissue. When the light emitted from the fluorescent material has the predetermined wavelength ranging from 560 nm to 605 nm (yellow light), the light of the fluorescent material may reduce fine wrinkles, and boost collagen synthesis so as to restore skin firmness. When the light emitted from the fluorescent material has the predetermined wavelength ranging from 605 nm to 625 nm (orange light), the light of the fluorescent material may improve metabolism of skin cells and gradually increase energy of skin cells. When the light emitted from the fluorescent material has the predetermined wavelength ranging from 625 nm to 780 nm (red light), the light of the fluorescent material may induce the epidermal cells to secrete collagen so as to rejuvenate skin.

In this embodiment, the light source used in the disclosure is a broad-band light source which includes not only visible light, but also UV light having a wavelength less than 380 nm and near-infrared light having a wavelength between 780 nm and 1100 nm. The UV light may cause skin injuries, and the near-infrared light may cause skin aging.

In order to filter out the UV light and the near-infrared light emitted from the light source, the barrier unit 3 is disposed on and in contact with the fluorescent layer 23 of the main body 2 so as to block the UV light and the near-infrared light from entering into the fluorescent layer 23 and the base layer 21, thereby preventing the skin from exposure to the UV light and the near-infrared light.

In certain embodiments, the barrier unit 3 includes a first filter 31 for filtering out the UV light with the wavelength less than 380 nm and a second filter 32 for filtering out the light with the wavelength between 780 nm and 1100 nm. In this embodiment, the first filter 31 is directly disposed on the fluorescence layer 23, and the second filter 32 is disposed on and in contact with the first filter 31 opposite to the fluorescent layer 23. The sequence of the first and second filters 31, 32 has no particular limits, the second filter 32 may be directly disposed on the fluorescence layer 23, and the first filter 32 may be disposed on and in contact with the second filter 32 opposite to the fluorescent layer 23 as long as the light of the light source that has the wavelength less than 380 nm or between 780 nm and 1100 nm may be blocked from entering into the fluorescent layer 23 and the base layer 21.

Furthermore, if the light source is not the broad-band light source, the first and second filters 31, 32 may be optionally formed based on the wavelength of the light emitted from the light source. For example, if the light emitted from the light source does not include UV light, the first filter 31 may be dispensed with.

Materials of the first and the second filters 31, 32 have no particular limits as long as they can block the light having a wavelength less than 380 nm and between 780 nm and 1100 nm, respectively. In this embodiment, the first filter 31 is made from a UV-absorbing material, and the second filter 32 is made from a nano ceramic material. In certain embodiments, the nano ceramic material may absorb near-infrared light and emit far-infrared light, so that the cosmetic product of the disclosure may be heated, and the skincare preparation 22 may be easier to be absorbed by the skin of a user.

In this embodiment, the cosmetic product is prepared by sequentially printing the fluorescent layer 23, the first filter 31 and the second filter 32 on the base layer, followed by adsorbing the skincare preparation 22 in the base layer 21.

In this disclosure, the cosmetic product may be a facial mask, a foot mask, an eye mask, a neck mask, or a breast mask. When the base layer absorbed with skincare preparation 22 is in contact with the user's skin and the cosmetic product is illuminated by the light source, the light emitted from the light source enters the second filter 32 which filters out the light having a wavelength between 780 nm and 1100 nm, then enters the first filter 31 which filters out the light having a wavelength less than 380 nm, and finally enters the fluorescent layer 23. The fluorescent material of the fluorescent layer 23 is then excited and emits light with the predetermined wavelength so as to provide the desired effects to the user's skin.

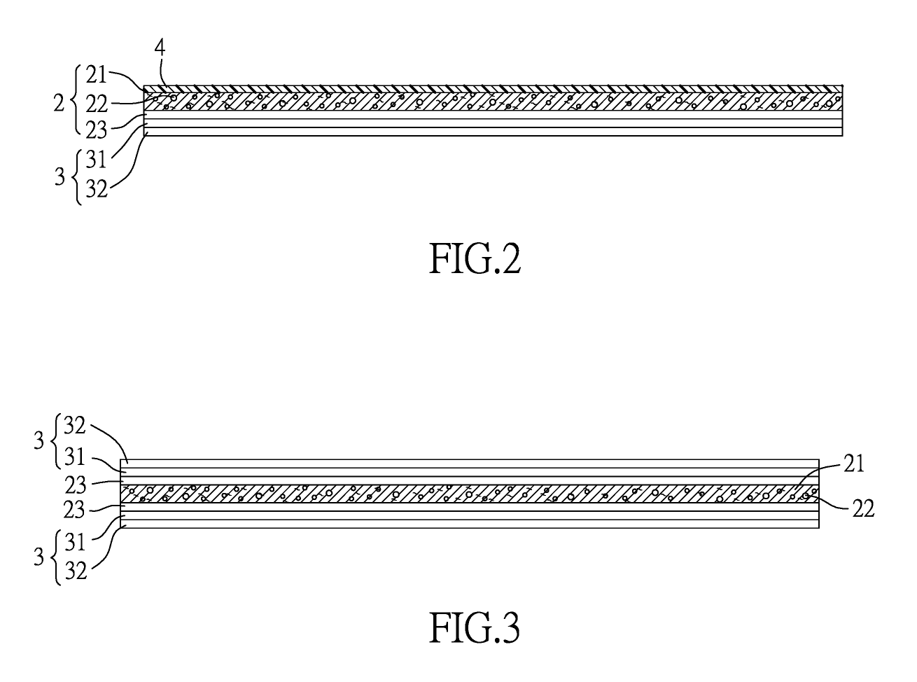

Referring to FIG. 2, a second embodiment of the cosmetic product according to the disclosure differs from the first embodiment in that the cosmetic product further includes a protective layer 4. The protective layer 4 is removably disposed on and in contact with the base layer 21 of the main body 2 and is made from a liquid-impermeable material. In certain embodiments, the protective layer 4 is made from a plastic material. The protective layer 4 may prevent the skincare preparation 22 from being damaged or contaminated by the external environment. Before using the cosmetic product of the disclosure, the protective layer 4 has to be removed from the main body 2.

Referring to FIG. 3, a third embodiment of the cosmetic product according to the disclosure differs from the first embodiment in that the cosmetic product of the third embodiment includes two of the fluorescent layers 23 respectively disposed on opposite sides of the base layer 21, and two of the barrier units 3 respectively disposed on the fluorescent layers 23. The cosmetic product of the third embodiment is prepared by: (a) immersing the base layer 21 into a first solution containing the fluorescent material to form the fluorescent layers 23 on the opposite sides of the base layer 21, (b) immersing the base layer 21 with the fluorescent layers 23 into a second solution containing the UV-absorbing material to form the first filter 31 on each of the fluorescent layers 23; (c) immersing the base layer 21 with the fluorescent layers 23 and the first filters 31 into a third solution containing the nano ceramic material to form the second filter 32 on each of the first filters 31; and (4) immersing the base layer 21 with the fluorescent layers 23 and the first and second filters 31, 32 into a fourth solution containing the skincare preparation 22, so that the skincare preparation 22 penetrates into the base layer 21 through the barrier units 3 and the fluorescent layers 23 and is absorbed by and retained in the base layer 21, thereby obtaining the cosmetic product.

In conclusion, with the fluorescent layer 23, the cosmetic product can emit light with the predetermined wavelength so as to provide desired cosmetic effects to the user's skin. Furthermore, with the barrier unit 3, the light emitted from the light source and having the wavelength less than 380 nm or between 780 nm and 1100 nm can be filtered out.

In the description above, for the purposes of explanation, numerous specific details have been set forth in order to provide a thorough understanding of the embodiment(s). It will be apparent, however, to one skilled in the art, that one or more other embodiments maybe practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A cosmetic product, comprising:
a main body including
a base layer,
a skincare preparation that is dispersed in or formed on the base layer, and
a fluorescent layer that is disposed on said base layer and that includes a fluorescent material emitting light with a predetermined wavelength after being excited by a light source; and
a barrier unit disposed on said fluorescent layer of said main body for blocking light emitted from said light source and having a wavelength less than 380 nm or between 780 nm and 1100 nm from entering into said fluorescent layer and said base layer.

2. The cosmetic product of claim 1, wherein said barrier unit includes a first filter for filtering out the light having a wavelength less than 380 nm from the light source.

3. The cosmetic product of claim 2, wherein said barrier unit further includes a second filter for filtering out the light having a wavelength between 780 nm and 1100 nm from the light source.

4. The cosmetic product of claim 3, wherein said first filter is disposed on said fluorescent layer, and said second filter is disposed on said first filter opposite to said fluorescent layer.

5. The cosmetic product of claim 1, further comprising a protective layer removably disposed on said base layer of said main body and made from a liquid-impermeable material.

6. The cosmetic product of claim 1, wherein said base layer is made from a material selected from the group consisting of woven fabric and non-woven fabric.

7. The cosmetic product of claim 3, wherein said first filter is made from a UV-absorbing material.

8. The cosmetic product of claim 3, wherein said second filter is made from a nano ceramic material.

9. The cosmetic product of claim 5, wherein said protective layer is made from a plastic material.

10. The cosmetic product of claim 1, wherein the light emitted from the fluorescent material has the predetermined wavelength selected from the group consisting of 450 nm to 495 nm, 495 nm to 560 nm, 560 nm to 605 nm, 605 nm to 625 nm, 625 nm to 780 nm, and combinations thereof.

11. The cosmetic product of claim 1, wherein the cosmetic product is a facial mask, a foot mask, an eye mask, a neck mask, or a breast mask.
